SEQUENCE LISTING (2 sequences)

SEQ ID NO: 1 - DNA, 4717 bp, Arabidopsis thaliana
atggatacgatgcgaatctccggtgtatcaaccggagctgaggttttaatacaatgcaattccttatcaagcctcgtttctcgtcgttgcgacgacggaaaatggcgaacgagaatgtttccggcgagaaacagagacttgcgtccatcgccgacgagaagatcctttttgtcggtgaaatctatctctagcgaaccgaaagccaaagtaaccgacgcagttctcgattccgaacaaggtctcattctaatacttgctttctaataagaattagggtacggaatttgaattttatagtgaatgttgtgaagtaactgattcgtattccttgggattttgtttttgtgttgattgattttcagaagtgtttattagctcgatgaatccgtttgcgccagatgctgcttcggtagcttcgagtatcaagtaccacgcggagtttacgccattgttttcaccggagaagtttgagttgccaaaggcgttctttgcgactgcgcaaagtgttagagatgctttgatcatgaattggaatgcaacttatgagtattacaacagagtgaatgtgaaacaagcgtattatttgtcaatggagtttttgcaggttttggtttttactcatttctttgagtgattttgttcttggttgttatctaactattttacattgtagggtagagccttatcgaatgccgtgggtaaccttgggcttaatagcgcttatggtgatgctttgaagaggcttggttttgatttggaaagcgtggctagtcaggtgagttgttaaccatgttgattattatgcattaaccgatgtttattactaacagacgtcttagagatgatcgtctttgcgagtctattgtttggttttacagagctgttatcttctttatatgtactgagatgctagatacttcacttccattttgtaggagccagatcctgcacttgggaatggtggactcgggagacttgcctcgtgttttttggattccatggcaactttgaattatccggcttggggttatggacttagatacaagtatggcttgttcaaacagagaattacaaaagatggacaggaggaagctgcagaagattggcttgaggtcttattctcttattcttttctcatacagcgtttgctattgaacagtatttcctaatttgtactctcttgtagcaatgctgagcagtggacatgtttattggcttacctgtttctttcagctaagcaatccttgggaaatagtcagaaatgatgtctcatatcctattaagttctatgggaaagtggtttttggatcagatggtaagaaacggtggattggtggagaagacattgttgctgttgcttatgatgttcctatacctggttataaaactaagacaactatcaatctgcggctctggtcaacaaaagctccttccgaagattttgatttatcttcatataactctgggaagcatactgaggcagcagaagctctattcaacgctgaaaaggtttgtatcttcattaagtttcatttaaagttgctttcacaattttgttttttcgaccatgatctatttacaagatccttctagtaattggaatagtgcatatatctttaaaattgagtgagaaccagcagaaatgaatatgttatcacagagagattagtcttgcgtcacttgtgcttgtttatataacgagcttttgatgtgtatatactgaaaagtggttgttttcttcccttccctcctgatggaattagatttgcttcgtgctttaccccggagatgagtcaactgaaggaaaggctcttcgtctgaagcaacaatacactctgtgctcagcctcgctacaagatatcgtagcacgttttgagacaaggtctggaggaaacgtcaactgggaagaatttccagagaaggttgcagtgcagatgaatgacactcaccctaccctatgcattcctgagctaatgaggattctaatggatttaaaaggactaagctgggaagacgcttggaaaatcacacaaaggtactaaaaatgactgaactaattgtcgggcatgctacatatgtgtctatttgttcctatatttagtctctggtgcttgtcccaaataaaagatagtttacaagaatgaaacctgcaacgtgtttctcaaaagttaataatttttttaggactgtggcatacacaaaccatacagtcttgcctgaggcactggagaagtggagtttagaactcatggagaaattgcttcctcgtcatgtggagattatcgaaaagattgatgaggaggtcatccctgaacaacatatcaaatgtctcttctatttttttcatatcgggtctaatttgtactttcatgtattgcagctagttcgcacaattgtttcagagtatggcaccgcggatcctgacttacttgaagaaaaactgaaggcaatgaggatcttggaaaatgtcgagttgccttctgcctttgcagatgtgatcgtgaagccggtgaacaaaccagttactgcaaaagatgctcaaaatggcgtgaaaacggaacaagaagaggaaaaaactgctggagaggaagaggaagacgaagttatcccagaaccaacagtagaaccccccaagatggtccgtatggccaaccttgctgttgtgggtggtcatgctgtaaatggcgttgcagagatacacagtgaaatagtgaagcaggacgtgtttaatgatttcgtacaggtaaacattctaactagtgaagcatgatgctataaaatgctctacagggaagaacacaactctcatcgttcaatattctatattttttgcagttgtggccagaaaaatttcagaacaaaacaaatggagtaacaccaaggcgatggattcgtttttgcaacccatatttaagtgatattataactaactggataggcacagaagactgggtcttaaataccgaaaaggttgcggaactaagaaaggtatgtactttatcagattcaatgttgtttcacatgctgttatctttattgggcgacattggttatcattgtttggtctttctccagtttgcagataatgaagatctccaatctgagtggagggcagcaaagaagaagaacaagttgaaggttgtatcacttatcaaggaaagaactggatatactgtcagccccgatgcaatgttcgacattcaggtcagttccaatggatcttggttacttttagattgatgagttgtttgcttgggtttttcggtttgagaagtcctttacgcaactctgagtagcttatgtagattcttttctttttgcattgaaaactttttgcagatcaagcgtatacatgagtacaagcgacaactgctaaatatcttgggaattgtttaccgctacaaaaagatgaaggaaatgagtgctagtgagagagagaaagcatttgttccaagagtttgcatatttgggggaaaagcatttgccacatatgtgcaagctaagagaattgttaaatttatcacagatgttgcgtctacaattaaccatgatccagaaataggtgacctccttaaggtatatatctacttacgttcttgtattagtcgtattctcaagcgtataacggaaaatctgcaataattatctggtttttgcatctgtggagattggcacttactaattagaagtgttaactaaacatgtaggttatctttgttcctgattacaatgtcagtgttgctgaattgctcattccagcaagtgagctttctcagcacatcaggtaaaaacttctttggcttagtcacattatagtttttggtcacaactccatgaagttaaaatattgaaattgagataaccggtaaaccatgaactggactagtttctctttttttcataagaactttagaaacaaatcctgacacaaggaacaatatgtttcggttacatttatgaaaggttataatcaatggcactcatactttttgctggagactaagagtttctctctgcagtactgctgggatggaagctagtgggacaagcaacatgaaattttcgatgaacggttgcgttttgattggaaccttggatggggcgaatgtcgagattagagaagaagttggagaagaaaatttcttcctctttggtgccaaagctgatcagattgtgaacctcaggaaggagagagcagagggaaaggtatatactatttgaagagttaaccttaccatgcttctgttttagcatcaacaagaatttgatttttgacctggctcttggcattccagtttgttcccgatcctacttttgaagaagtcaagaagttcgttggaagcggcgtctttggctcaaatagctatgatgaactaatcggctctttggaaggaaacgaaggctttggacgagcggattacttcctagttggcaaagactttcctagttacatcgaatgccaagaaaaagtcgacgaggcataccgagaccagaaagtaagtactaatgcattttctttgaacatcaagctaataatgttgactaaaatatgaaacttactcaaatatcaaaccttgaaattgctgttaaatgattacagagatggacgagaatgtcaataatgaacacagcaggttcattcaagtttagcagtgaccggacgatccacgaatacgccaaagacatatggaatattaagcaagtggaacttccatga

SEQ ID NO: 2 - DNA, 10870 bp, Arabidopsis thaliana
  misc_feature (2040)..(8192): T-DNA sequence
atggatacgatgcgaatctccggtgtatcaaccggagctgaggttttaatacaatgcaattccttatcaagcctcgtttctcgtcgttgcgacgacggaaaatggcgaacgagaatgtttccggcgagaaacagagacttgcgtccatcgccgacgagaagatcctttttgtcggtgaaatctatctctagcgaaccgaaagccaaagtaaccgacgcagttctcgattccgaacaaggtctcattctaatacttgctttctaataagaattagggtacggaatttgaattttatagtgaatgttgtgaagtaactgattcgtattccttgggattttgtttttgtgttgattgattttcagaagtgtttattagctcgatgaatccgtttgcgccagatgctgcttcggtagcttcgagtatcaagtaccacgcggagtttacgccattgttttcaccggagaagtttgagttgccaaaggcgttctttgcgactgcgcaaagtgttagagatgctttgatcatgaattggaatgcaacttatgagtattacaacagagtgaatgtgaaacaagcgtattatttgtcaatggagtttttgcaggttttggtttttactcatttctttgagtgattttgttcttggttgttatctaactattttacattgtagggtagagccttatcgaatgccgtgggtaaccttgggcttaatagcgcttatggtgatgctttgaagaggcttggttttgatttggaaagcgtggctagtcaggtgagttgttaaccatgttgattattatgcattaaccgatgtttattactaacagacgtcttagagatgatcgtctttgcgagtctattgtttggttttacagagctgttatcttctttatatgtactgagatgctagatacttcacttccattttgtaggagccagatcctgcacttgggaatggtggactcgggagacttgcctcgtgttttttggattccatggcaactttgaattatccggcttggggttatggacttagatacaagtatggcttgttcaaacagagaattacaaaagatggacaggaggaagctgcagaagattggcttgaggtcttattctcttattcttttctcatacagcgtttgctattgaacagtatttcctaatttgtactctcttgtagcaatgctgagcagtggacatgtttattggcttacctgtttctttcagctaagcaatccttgggaaatagtcagaaatgatgtctcatatcctattaagttctatgggaaagtggtttttggatcagatggtaagaaacggtggattggtggagaagacattgttgctgttgcttatgatgttcctatacctggttataaaactaagacaactatcaatctgcggctctggtcaacaaaagctccttccgaagattttgatttatcttcatataactctgggaagcatactgaggcagcagaagctctattcaacgctgaaaaggtttgtatcttcattaagtttcatttaaagttgctttcacaattttgttttttcgaccatgatctatttacaagatccttctagtaattggaatagtgcatatatctttaaaattgagtgagaaccagcagaaatgaatatgttatcacagagagattagtcttgcgtcacttgtgcttgtttatataacgagcttttgatgtgtatatactgaaaagtggttgttttcttcccttccctcctgatggaattagatttgcttcgtgctttaccccggagatgagtcaactgaaggaaaggctcttcgtctgaagcaacaatacactctgtgctcagcctcgctacaagatatcgtagcacgttttgagacaaggtctggaggaaacgtcaactgggaagaatttccagagaaggttgcagtgcagatgaatgacactcaccctaccctatgcattcctgagctaatgaggattctaatggatttaaaaggactaagctgggaagacgcttggaaaatcacacaaaggtactggcaggatatatgccaacgtaaaaatgagggcaatcgattgtactgaatcggattttcaagggtctggccaaaactattccgtgggcacctggcacacgccctggagtccggcccgtttccagttgagggttgtctacgcttagatgagaaggaaagttgtccaagacgaatcccagtgtcctattaccaatagccgacggtatcgataagcttgatgtacatggtcgataagaaaaggcaatttgtagatgttaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccatcggatcctagacgcgtgagatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatccgatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatggcctttgattcagtaggaactactttcttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttgatgagacctgccgcgtaggcctctctaaccatctgtgggtcagcattctttctgaaattgaagaggctaatcttctcattatcggtggtgaacatggtatcgtcaccttctccgtcgaactttcttcctagatcgtagagatagagaaagtcgtccatggtgatctccggggcaaaggagatccgtcaattccgattcattaatgcagctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatcaaagatccggcccatgatcatgtggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggggctcgatcccctcgatcgaattcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgagctcaattccccaccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgaaatattcccgtgcacttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttggcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttaaaaatgactgaactaattgtcgggcatgctacatatgtgtctatttgttcctatatttagtctctggtgcttgtcccaaataaaagatagtttacaagaatgaaacctgcaacgtgtttctcaaaagttaataatttttttaggactgtggcatacacaaaccatacagtcttgcctgaggcactggagaagtggagtttagaactcatggagaaattgcttcctcgtcatgtggagattatcgaaaagattgatgaggaggtcatccctgaacaacatatcaaatgtctcttctatttttttcatatcgggtctaatttgtactttcatgtattgcagctagttcgcacaattgtttcagagtatggcaccgcggatcctgacttacttgaagaaaaactgaaggcaatgaggatcttggaaaatgtcgagttgccttctgcctttgcagatgtgatcgtgaagccggtgaacaaaccagttactgcaaaagatgctcaaaatggcgtgaaaacggaacaagaagaggaaaaaactgctggagaggaagaggaagacgaagttatcccagaaccaacagtagaaccccccaagatggtccgtatggccaaccttgctgttgtgggtggtcatgctgtaaatggcgttgcagagatacacagtgaaatagtgaagcaggacgtgtttaatgatttcgtacaggtaaacattctaactagtgaagcatgatgctataaaatgctctacagggaagaacacaactctcatcgttcaatattctatattttttgcagttgtggccagaaaaatttcagaacaaaacaaatggagtaacaccaaggcgatggattcgtttttgcaacccatatttaagtgatattataactaactggataggcacagaagactgggtcttaaataccgaaaaggttgcggaactaagaaaggtatgtactttatcagattcaatgttgtttcacatgctgttatctttattgggcgacattggttatcattgtttggtctttctccagtttgcagataatgaagatctccaatctgagtggagggcagcaaagaagaagaacaagttgaaggttgtatcacttatcaaggaaagaactggatatactgtcagccccgatgcaatgttcgacattcaggtcagttccaatggatcttggttacttttagattgatgagttgtttgcttgggtttttcggtttgagaagtcctttacgcaactctgagtagcttatgtagattcttttctttttgcattgaaaactttttgcagatcaagcgtatacatgagtacaagcgacaactgctaaatatcttgggaattgtttaccgctacaaaaagatgaaggaaatgagtgctagtgagagagagaaagcatttgttccaagagtttgcatatttgggggaaaagcatttgccacatatgtgcaagctaagagaattgttaaatttatcacagatgttgcgtctacaattaaccatgatccagaaataggtgacctccttaaggtatatatctacttacgttcttgtattagtcgtattctcaagcgtataacggaaaatctgcaataattatctggtttttgcatctgtggagattggcacttactaattagaagtgttaactaaacatgtaggttatctttgttcctgattacaatgtcagtgttgctgaattgctcattccagcaagtgagctttctcagcacatcaggtaaaaacttctttggcttagtcacattatagtttttggtcacaactccatgaagttaaaatattgaaattgagataaccggtaaaccatgaactggactagtttctctttttttcataagaactttagaaacaaatcctgacacaaggaacaatatgtttcggttacatttatgaaaggttataatcaatggcactcatactttttgctggagactaagagtttctctctgcagtactgctgggatggaagctagtgggacaagcaacatgaaattttcgatgaacggttgcgttttgattggaaccttggatggggcgaatgtcgagattagagaagaagttggagaagaaaatttcttcctctttggtgccaaagctgatcagattgtgaacctcaggaaggagagagcagagggaaaggtatatactatttgaagagttaaccttaccatgcttctgttttagcatcaacaagaatttgatttttgacctggctcttggcattccagtttgttcccgatcctacttttgaagaagtcaagaagttcgttggaagcggcgtctttggctcaaatagctatgatgaactaatcggctctttggaaggaaacgaaggctttggacgagcggattacttcctagttggcaaagactttcctagttacatcgaatgccaagaaaaagtcgacgaggcataccgagaccagaaagtaagtactaatgcattttctttgaacatcaagctaataatgttgactaaaatatgaaacttactcaaatatcaaaccttgaaattgctgttaaatgattacagagatggacgagaatgtcaataatgaacacagcaggttcattcaagtttagcagtgaccggacgatccacgaatacgccaaagacatatggaatattaagcaagtggaacttccatga

(19) United States
(12) Patent Application Publication
(10) Pub. No.: US 2007/0209088 A1
(43) Pub. Date: Sep. 6, 2007

(54) Method for Improving Plants

(76) Inventors: Christophe D'Hulst, Wattrelos (FR); Veronique Planchot, Nantes (FR); Manash Chatterjee, Cambridge (GB)

Correspondence Address: YOUNG & THOMPSON, 745 SOUTH 23RD STREET, 2ND FLOOR, ARLINGTON, VA, 22202, US

(21) Appl. No.: 10/594,526

(22) PCT Filed: Mar. 29, 2005
(86) PCT No.: PCT/FR05/00753

§ 371 Date: Nov. 27, 2006

(30) Foreign Application Priority Data
Mar. 29, 2004 (FR) 04 03242

(51) Int. Cl. C12N 15/82; A01H 5/10

(52) U.S. Cl. 800/284; 435/419; 800/298

(57) ABSTRACT

The invention relates to a method for improving the size of starchy grains and/or the content of starch in a plant or a part of a plant, whereby the gene of a starch phosphorylase in the cells of the plant is inactivated.

The present invention relates to a method for increasing the size of the starch grains produced in plants and/or for increasing the starch content of plants.

Starch is the energy storage polysaccharide in plants. It constitutes the main supply of calories in animal and human nutrition and is also a major source of vegetable raw material for non-food uses. Starch is composed of two separate polysaccharide fractions: amylose and amylopectin. Amylose, which represents the minor fraction of starch, consists of glucose residues joined together by α-1,4 bonds, and has less than 1% branching. Amylopectin, which represents the major fraction of starch, consists of glucose residues joined together by α-1,4 bonds, and has about 5% branching, comprising glucose residues bound to the main polymer by an α1,6 bond. The asymmetric distribution of the branching of amylopectin is responsible for the limitless growth of starch molecules and consequently of starch grains, and also accounts for most of the physicochemical properties of starch.

The biosynthesis of starch depends on a metabolic pathway with, as principal biochemical stages, the synthesis of ADP-glucose followed by the transfer of this precursor in position α-1,4 on a glucan by (ADP-glucose: 1,4-α-D-glucan 4-α-D-glucosyl)transferases, the polymer formed being made to branch by the action of the so-called branching enzymes: the 1,4-α-D-glucan 6-α-D-(1,4-α-D-glucano)-transferases.

The degradation of starch involves several enzymes, including α-amylase (endoamylase), β-amylase (exoamylase), amyloglucosidase, and alpha-glucan phosphorylase (starch phosphorylase).

The role of these various starch degrading enzymes has not been clearly established. For example, it has been reported that reduced expression of a phosphorylase in leaves, by antisense repression, did not have a significant influence on the accumulation of starch, in the potato (Sonnewald et al., 1995).

Since the antisense repression of α-glucan phosphorylase activity did not have a significant influence on the accumulation of starch in the leaves of transgenic potatoes, the authors concluded from this that the breakdown of starch is not catalysed by the phosphorylases.

U.S. Pat. No. 5,998,701 discloses that reduction of the content of phosphorylase in potato tubers leads to a substantial decrease in the accumulation of sugars, which can be utilized for prolonging the storage time of potato tubers.

U.S. Pat. No. 6,353,154 proposes, for its part, modifying the activity of starch phosphorylase in plants, in particular maize, with the aim of obtaining the synthesis of starch with a modified structure.

The inventors have now demonstrated that inactivation of the gene coding for a starch phosphorylase causes a significant increase in the size of the starch grains produced in the plants, as well as significantly increasing the amount of starch accumulated.

On this basis, the present invention provides a method for increasing the size of the starch grains of a plant or of a plant part, in which the gene of a starch phosphorylase is inactivated in the cells of the plant.

This method is particularly advantageous for increasing yields in the extraction and purification of starch on an industrial scale. In fact, the smallest starch grains are generally lost during washings in the course of the processes of extraction and purification. Increase in the size of the grains can avoid the loss of a proportion of the starch grains.

The present invention also provides a method for increasing the starch content of a plant or plant part, in which the gene of a starch phosphorylase in the cells of the plant is inactivated.

It must be understood that increase in size of the starch grains and increase in starch content are not necessarily related, that is, a priori, increase in starch content does not necessarily involve an increase in the size of the starch grains, and vice versa.

The present application shows that there is interaction between starch phosphorylase, starch synthase, and the branching enzymes. Phosphorylase, possibly by interaction with a glycogenin (WO 03/014365), would prime the initiation of starch by supplying the appropriate primer for the branching enzymes and for starch synthase.

Without being bound to this theory, a hypothesis can be put forward to explain the increase in average size of the starch grains in plants in which starch phosphorylase has been inactivated. According to this theory, owing to inactivation of phosphorylase, only starch synthase (notably SS 5 in Arabidopsis, SS I in maize) would be able to interact with glycogenin and initiate starch synthesis. Weaker interaction with glycogenin, or also delayed expression, would lead to a delay in the initiation of starch synthesis, and therefore of the number of granules produced (initiated). As a smaller number of starch molecules is initiated but the substrates necessary for starch synthesis are present at the same level, coarser grains are obtained since the same amount of substrate is used for a smaller number of granules.

The invention also provides a method of obtaining plants or plant parts producing starch grains of increased size, said method comprising the inactivation of the gene of a starch phosphorylase in the cells of the plant.

The invention further provides a method for the production of plants, plant tissues or plant parts with higher starch content, said method comprising the inactivation of the gene of a starch phosphorylase in the cells of the plant.

The term “plant tissue” refers to any tissue of a plant, in a plant or in a culture. This term includes whole plants, plant cells, plant organs, plant seeds, protoplasts, calluses, cell cultures and all other plant cells organized as a functional and/or structural unit.

The invention also relates to any plant tissue that can be obtained by the method according to the invention as well as transgenic plants containing it.

Moreover, the seeds from the plants obtained according to one of the methods of the invention, characterized in that they have an increased size, and/or a modified starch content, fall within the scope of the present invention.

The “starch phosphorylases”, also known by the name “alpha-glucan phosphorylases”, have been described in numerous plants, for example broad bean, potato (Swissprot P04045), beet, spinach, maize (WO 98/40503), pea, as well as rice (EMBL access No. D23280 or Q9AUV8), and wheat (EMBL AAQ73181).

The genomic sequence (locus designated AtPHO-1) coding for the starch phosphorylase of Arabidopsis thaliana is given in the appendix (SEQ ID No 1).

The starch phosphorylase is directed to the cell plastid.

A person skilled in the art knows how to identify the phosphorylases that are to be inactivated, for example by comparing sequences between SEQ ID No1 with sequences of other species using sequence-comparing software such as Blast (www.ncbi.nlm.nih.gov) and the FastDB program with the default parameters. These algorithms are presented in Current Methods in Sequencing and Synthesis, Methods and Applications, pages 127-149, 1988, Ala. R. Liss, Inc., which is incorporated in the description by reference. Another possible method is based for example on selective hybridization in conditions of high stringency as defined in Sambrook et al. Molecular Cloning, A Laboratory Manual (Cold Spring Harbor Press, 1989) in paragraphs 11.1 to 11.61. In particular it may relate more particularly to allelic forms of the aforementioned enzymes. The phosphorylases to be inactivated are preferably addressed to the plastid, i.e. directed towards the plastid. A person skilled in the art is able to identify the motif on the sequence corresponding to the peptide for addressing to the plastid. This can be done using for example the Genoplante Predotar software (Small I. et al., 2004, Proteomics, vol 4. (6) 1581-1590 and accessible on the site http://www.genoplante.com).

The expression “higher starch content” signifies that the transgenic plant obtained supplies a larger amount of starch than an untransformed plant of the same species.

“Inactivation of the starch phosphorylase gene” signifies that the gene is switched off, i.e. it no longer or practically no longer permits expression of an active starch phosphorylase protein, the protein being no longer or practically no longer expressed, or is expressed in a non-functioning mutated form, incapable of exercising its enzymatic properties.

The gene can be inactivated by any means by a person skilled in the art (see Thorneycroft et al., 2001), in particular by gene interruption, or by gene silencing.

According to a preferred embodiment, a mutation is introduced in the gene coding for starch phosphorylase, which switches off this gene, i.e. it becomes incapable of expressing the enzyme, or the enzyme produced is inactive.

In particular, the mutation can comprise insertion of nucleotide(s), for example between exon 6 and intron 6 of the starch phosphorylase gene.

Gene silencing can thus be achieved by insertion of T-DNA.

Thus, the sequence SEQ ID No 2 shows the starch phosphorylase gene of Arabidopsis thaliana in which a T-DNA sequence has been inserted.

The invention also relates to the use of the polynucleotide sequence SEQ ID No2 for producing a plant with modified size of the starch grains and/or modified starch content. The plant obtained according to the invention is selected from potato, broad bean, beet, spinach, pea, wheat, maize or rice.

T-DNA has been used as a mutagen since the end of the 1980s. In Arabidopsis, which does not possess endogenous transposons having activity that permits insertional mutagenesis, it has been used in preference to transposons. The soil bacterium Agrobacterium is capable of transferring a piece of its DNA, the T-DNA, into the nuclear genome of plant cells. This property is very useful for switching off genes by insertional mutagenesis. The only elements required are repetitions of 24 base pairs, the border sequences, which delimit the region to be transferred. The increase in efficacy of transformation techniques facilitated the development of reverse genetics.

Infiltration of whole plants under vacuum made it possible to increase the efficacy of transformation (4 to 5 transformants per plant treated) as well as the reproducibility. Recently, the method has been further simplified with the appearance of the “floral dip”. The flowers are simply dipped in a suspension of Agrobacterium in the presence of a surfactant, Silwet L-77, and sucrose. With these various methods, all the transformants obtained are hemizygous for T-DNA, which suggests delayed transformation in the course of floral development. The target of transformation has been identified as being the developing ovule. Mutations that are lethal in the homozygous state are maintained in the population in the form of heterozygous plants. Two insertions are obtained on average per plant. Segregation analyses show that 57% of the transformants contain 1 insertion locus, 25% 2 loci, 8% 3 loci and 2% more than 3. Molecular analysis of labelled mutants shows that these insertions are made randomly, are stable, are maintained in the progeny and there is little insertion bias.

The endogenous starch phosphorylase gene can also be switched off by mutagenesis of plant cells, for example by UV irradiation, by a chemical mutagen, or by insertion of transposons.

The transposable elements have the capacity to disturb the expression of genes in which they are inserted and to generate deletions, rearrangements, and mutations at the target locus.

Transposons were the first insertional mutagens used in maize, then in petunia and Antirrhinum. In contrast to T-DNA, the transposon can be excised from the disrupted gene in the presence of a transposase. The high frequency of reversion of the resultant mutation confirms that it is induced by the transposon. The remobilization of transposons also makes it possible to generate mosaics: a homozygous mutant which carries an active transposase will have somatic sectors which have lost the transposon Ds and restored the function of the gene. This makes it possible to determine the site of action of a gene in combination with its expression template. However, for transposons of type Ac/Ds, most of the transposition events occur at genetically linked sites. If there is a transposable element near a gene of interest, it can therefore be remobilized to be inserted in the gene or close by (Ito et al., 1999). It is thus possible to effect local mutagenesis in a region of particular interest.

A technique of mutagenesis by transposons which can be used advantageously is mutagenesis by Mutator transposon confirmed by screening in reverse genetics (Bensen et al., 1995; Das et al., 1995). This technique employs stages comprising crossing a “Mutator” line with hybrids of the plants of interest then screening the F1 plants obtained by PCR with a primer specific to the transposons and a primer specific to the nucleotide sequence coding for starch phosphorylase. The F2 seeds obtained from the screened F1 plants then produce plants, whose phenotype is then analysed.

Another method for switching off the starch phosphorylase gene is the local injection of double-stranded RNA (RNA interference: RNAi) (Fire, 1999). The double-stranded RNAs are cleaved into small sense and antisense RNAs of about 22 nucleotides which will target the degradation of the endogenous homologous mRNAs (Zamore et al., 2000). The constitutive expression of double-stranded RNA by a transgene employing inverted repeat sequences under the control of the 35S promoter makes it possible to obtain effective inactivation in the whole plant, even in the meristem (Waterhouse et al., 1998). This strategy is very effective throughout plant development.

The gene of an endogenous starch phosphorylase can moreover be switched off by a method comprising the stages of:

a) supplying an expression vector comprising an antisense nucleotide sequence of the gene coding for said endogenous starch phosphorylase;

b) transforming a plant cell with said expression vector;

c) regenerating the plant from the cell transformed in stage b, said transgenic plant thus obtained having starch grains of increased size, with a higher starch content.

Another possibility for reducing the activity of starch phosphorylase in the plant cells is to express ribozymes, which are RNA molecules that act as enzymes specifically catalysing the cleavage of transcripts coding for starch phosphorylase, by techniques known by a person skilled in the art (EP 321 021).

It is also possible to obtain a plant with altered expression of starch phosphorylase by the so-called “transwitch” method described in WO90/12084.

The starch phosphorylase gene can also be switched off by infecting the plants with recombinant viruses into which a portion of the coding sequence or of the promoter of the gene to be switched off has been inserted (virus-induced gene silencing or VIGS) (Ratcliff et al., 2001). To explain this phenomenon, it is thought that the viral molecules of positive and negative polarity produced in the course of the cycle of viral replication are recognized as double-stranded RNA and degraded into small sense and antisense RNAs of 22 nucleotides which in their turn trigger the degradation of the endogenous homologous mRNAs. However, only the endogenous mRNAs are completely degraded, whereas the viral RNAs are still detectable. The presence of small RNAs of 22 nucleotides derived from the viral RNA suggests that the viruses which induce VIGS are also capable of resisting it. The advantages of this method are above all its simplicity and speed in use. Moreover, it is sufficient to clone 23 base pairs of a gene in the virus for specifically targeting its inactivation.

Construction of the expression vectors used (for example carrying an antisense sequence of the endogenous starch phosphorylase gene) or of the iRNAs is within the capacity of a person skilled in the art using standard methods.

The transformation of plant cells can be effected by transferring vectors or nucleic acids into the protoplasts, notably after incubation of the latter in a solution of polyethylene glycol in the presence of divalent cations (Ca²⁺).

The transformation of plant cells can also be effected by electroporation notably by the method described in the article by Fromm et al., 1986.

The transformation of plant cells can also be effected using a gene gun for bombardment, at very high velocity, of metallic particles coated with the DNA sequences of interest, thus delivering genes to the interior of the cell nucleus, notably by the technique described in Sanford's article (1988).

Another method of transformation of plant cells is cytoplasmic or nuclear micro-injection.

According to a particularly preferred embodiment of the method of the invention, the plant cells are transformed by biolistics, i.e. by bombardment, by means of a particle gun, of microparticles coated with the nucleotide sequences to be transferred (J. Finner, 1992).

According to another embodiment of the method of the invention, the plant cells are transformed by a vector according to the invention, said host cell being capable of infecting said plant cells by permitting the integration, in the genome of the latter, of the DNA sequences of interest initially contained in the genome of the aforementioned vector.

Advantageously, the aforementioned host cell used is Agrobacterium tumefaciens, notably by the method described in the article of An et al., 1986, or alternatively Agrobacterium rhizogenes, notably by the method described in the article of Jouanin et al., 1987.

Preferably, the transformation of the plant cells is accomplished by the transfer of the T region of the extrachromosomal circular, tumour-inducing plasmid Ti of Agrobacterium tumefaciens, using a binary system (Watson et al.).

To do this, two vectors are constructed. In one of these vectors, the T-DNA region has been removed by deletion, apart from the right and left edges, a marker gene being inserted between them to permit selection in plant cells. The other partner of the binary system is an auxiliary Ti plasmid, a modified plasmid which no longer has T-DNA but still contains the virulence genes vir, required for transformation of the plant cell. This plasmid is maintained in Agrobacterium.

Among the transcription terminators that can be used, we may mention the terminator polyA 35S of the cauliflower mosaic virus (CaMV), described in the article of Franck et al., (1980), or the terminator polyA NOS, which corresponds to the noncoding 3′ region of the gene of nopaline synthase of the Ti plasmid of Agrobacterium tumefaciens nopaline strain (Depicker et al., 1982).

Among the transcription promoters that can be used, we may mention notably:
the promoter 35S, or advantageously the constitutive double promoter 35S (pd35S) of the CaMV, described in the article of Kay et al., 1987;
the promoter PCRU of the radish cruciferin gene permitting expression of the associated sequences only in the seeds of the transgenic plant obtained;
the promoters PGEA1 and PGEA6 corresponding to the noncoding 5′ region of the genes of the seed storage protein, GEA1 and GEA6, respectively, of Arabidopsis thaliana (Gaubier et al., 1993) and permitting specific expression in the seeds;
the chimeric promoter super-promoter PSP (Ni M et al., 1995), comprising the fusion of a triple repetition of a transcriptional activator element of the promoter of the octopine synthase gene of Agrobacterium tumefaciens, of a transcriptional activator element of the promoter of the mannopine synthase gene and of the mannopine synthase promoter of Agrobacterium tumefaciens;
the actin promoter of rice followed by the actin intron of rice (PAR-IAR) contained in the pAct1-F4 plasmid described by Mc Elroy et al., 1991;
the promoter HMGW (High Molecular Weight Glutenin) of barley;
the promoter of the γzeine gene of maize (Pγzeine) contained in the pγ63 plasmid, and permitting expression in the albumen of maize seeds.

Among plant cells that can be transformed in accordance with the present invention, we may mention those of potato, broad bean, beet, spinach, pea, wheat, maize or rice.

The present invention also makes it possible to obtain a plant or plant part such as notably potato, wheat, maize or rice, producing starch grains of increased size, or a higher starch content.

By “plant part”, we mean notably the storage organs that are naturally rich in starch, such as seeds or tubers. By “plant part”, we also mean the cells of said plant.

The starch produced can be extracted according to the standard methods known by a person skilled in the art. The solubilization of starch is also known by a person skilled in the art and can be carried out by soaking and fractionation of the starch grains, or for example by heating. Alternatively, starch destructuring enzymes, such as the amylases, can be used.

The starch produced can also be used in many industries: the paper and cardboard industry, adhesives industry, textile industry, pharmaceutical industry (in the formulation of medicinal products), etc.

The starch produced can also be submitted to other modifications, in particular chemical modifications such as acid treatment, oxidation, esterification, etc. before it is used.

This starch can be used for the preparation of derivative products, notably of foodstuffs.

The following figures and examples illustrate the invention without limiting its scope.

### LEGENDS OF THE FIGURES

FIG. 1 is a schematic diagram representing the genome of Arabidopsis thaliana.

FIG. 2 is a graph showing the relative levels of accumulation of starch in the mutant line relative to the wild-type reference line (WS).

FIG. 3 compares profiles from spectrophotometric analysis of starch from the wild-type and mutant lines after steric exclusion chromatography on a sepharose CL-2B matrix.

FIG. 4 compares photographs of images obtained in the transmission electron microscope, of starch grains (magnification ×3000).

### EXAMPLES

1. Description of the Mutant Line:

The inventors investigated the phenotypes of a mutant line of Arabidopsis thaliana, produced by interruption of a starch phosphorylase gene (locus designated AtPHO-1).

This line (DDS72) is one of the 50 000 mutant lines produced by random insertion of T-DNA, as described by Balzuergue et al., 2001.

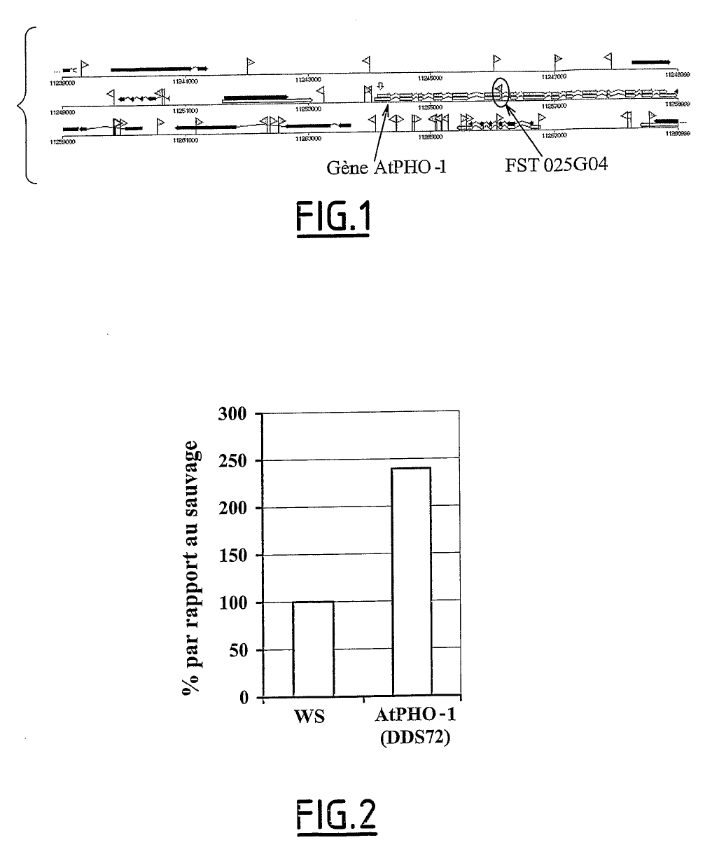

The mutant line DDS72 of Arabidopsis thaliana investigated has an insertion of T-DNA at the junction of exon 6 and intron 6 (cf. FIG. 1 and SEQ ID No 2).

2. Enzymological Analysis of the Mutant Line:

In order to determine the effect of insertion of T-DNA at the AtPHO-1 locus on the activity of starch phosphorylases, the inventors obtained zymograms from cellular extracts of various mutant and wild-type (WS) lines. The zymograms were obtained in two different conditions.

—Extraction of Proteins from the Leaves

The leaves are pulverized at 4° C. using a Polytron Blender in the following buffer: 50 mM NaH₂PO₄, 0.5 M NaCl. The pulverized material is centrifuged for 5 minutes at 13000 rpm at 4° C. and the supernatant containing the soluble proteins is recovered.

Polyacrylamide Gel Electrophoresis

The gels are produced using MiniProtean II electrophoresis chambers marketed by BioRAD (Richmond, Calif., USA). The gels have a thickness of 1.5 mm. The final concentration of monomer is 7.5% (w/v) for the separation gel, it also contains 0.45% of rabbit liver glycogen or 0.2% of potato starch. It is buffered with Tris/HCl 110 mM pH 7.2. The gel for final monomer concentration at 2.5% is buffered with Tris/H₃PO₄ 60 mM pH 7.3. The migration buffer used in electrophoresis is Glycine/Tris 40 mM pH 8.5.

10 μl of Tris/H₃PO₄ 60 mM pH 7.3 and 20 μl of loading buffer: sucrose 25% (w/v), bromophenol blue 0.001%, are added to 100 μg of protein extract.

Migration is carried out at 4° C. for 2 h 30 min at 15 mA and 250V. The gel is then equilibrated in Citrate/NaOH 100 mM pH 7.0 for 10 minutes before being incubated overnight at room temperature in citrate/NaOH 100 mM pH 7.0, glucose-1-phosphate 50 mM.

At this concentration, the phosphorylases function in the direction of synthesis of polysaccharides by adding a glucose residue at the non-reducing end of the available glycans by means of an α-1,4 bond. The activity is then detected by staining the gel with iodine.

It is the fast-migrating form (on glycogen or starch) which disappears completely in the mutant at the AtPHO-1 locus.

3. Effect of Mutation on the Storage Polysaccharide:

Extraction of Starch from the Leaves of Arabidopsis thaliana

The leaves of A. thaliana are taken at the end of a photoperiod then rinsed twice in a large volume of deionized water (in order to remove the unwanted debris). In ice, the material is pulverized in 15-25 ml of extraction buffer (MOPS 100 mM pH 7.2, EDTA 5 mM, ethylene glycol 10) using a Polytron Blender (tissue homogenizer) until a really homogeneous extract is obtained, without any intact tissue. The extract is passed 4×15 seconds in the “continuous” sonicator, immersing the tube in ice between each sonication. Centrifuge for 15 minutes at 3200 g and 4° C. The deposit is taken up in 20 ml of Percoll (Amersham Biosciences) at 90% and centrifuged for 40 minutes at 10000 g in a Corex glass tube. The surface debris and the supernatant are removed. The deposit of starch is rinsed five times with deionized water before being analysed.

Determination of the Starch

The starch is assayed using the Enzytec kit marketed by Diffchamb (Lyon, France). The glucans are digested with amyloglucosidase which hydrolyses the α-1,4 and α-1,6 O-glycoside bonds. The glucose molecules thus released are then phosphorylated in position 6 with a hexokinase. The glucose-6-phosphate produced is then oxidized to gluconate-6-P by a G6P dehydrogenase, reducing NADP to NADPH. This last-mentioned reaction is monitored with the spectrophotometer at 365 nm.

The quantity of starch determined is shown in Table 1:

TABLE 1

| | Quantity of starch |
|---|---|
| Line | (in mg/g of leaves) |
| WS (wild-type line) | 1.16 |
| AtPHO-1 (line | 2.78 |
| DDS72) | |

FIG. 2 shows the relative levels of accumulation of starch in the various lines relative to the wild-type reference line (WS).

The structure of the starch is then analysed by steric exclusion chromatography on a sepharose CL-2B matrix.

Fractionation of the Starch

Fractionation is carried out by steric exclusion chromatography on sepharose CL-2B matrix (Amersham-Biosciences, Sweden).

The column has an inside diameter of 0.5 cm and a height of 65 cm. Equilibrated in soda 10 mM, it has a flow rate of 12 ml/hour. Preparation of the starch sample is as follows: 1.5 mg of native starch is dissolved in 200 μl of DMSO 100% at 100° C. for 10 minutes. The polysaccharide is then precipitated with 4 volumes of pure ethanol at −20° C. for 30 min. After centrifugation at 5000 g for 5 minutes, the starch deposit is dissolved in 500 μl of soda 10 mM then deposited on the column. Fractions of 300 μl are analysed by iodine spectrophotometry.

Determination of λₘₐₓ of the Iodine-Polysaccharide Complex:

The length of the maximum absorbance of the complex formed by iodine with the polysaccharides is determined by spectrophotometry. 100 μg of starch is dissolved in dimethylsulphoxide (DMSO) 100% for 10 minutes at 100° C. This solution is then adjusted to 10% in DMSO. 100 μl of a solution of iodine 0.02% I₂ and 0.2% KI is added to 400 μl of this solution. The absorption spectrum is recorded between 400 and 700 nm.

The amounts of polysaccharides present in each fraction can also be determined using the Enzytec assay kit.

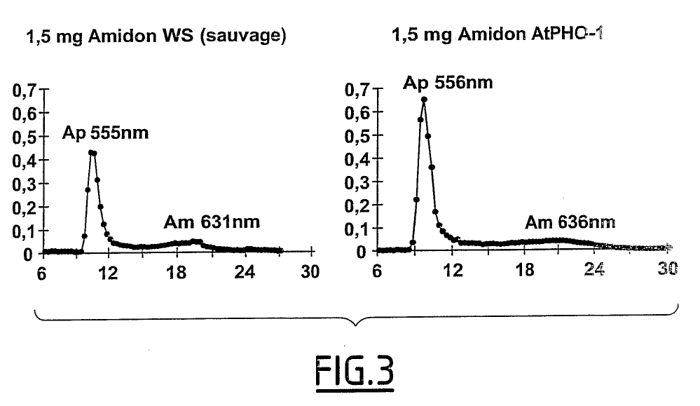

There does not seem to be a particular change in the structure of the starch from the mutant line AtPHO-1, if we compare the two profiles shown in FIG. 3.

4. Analysis of the Structure of the Starch Accumulated by the AtPHO-1 Line by Electron Microscopy:

Sample Preparation for Transmission Electron Microscopy

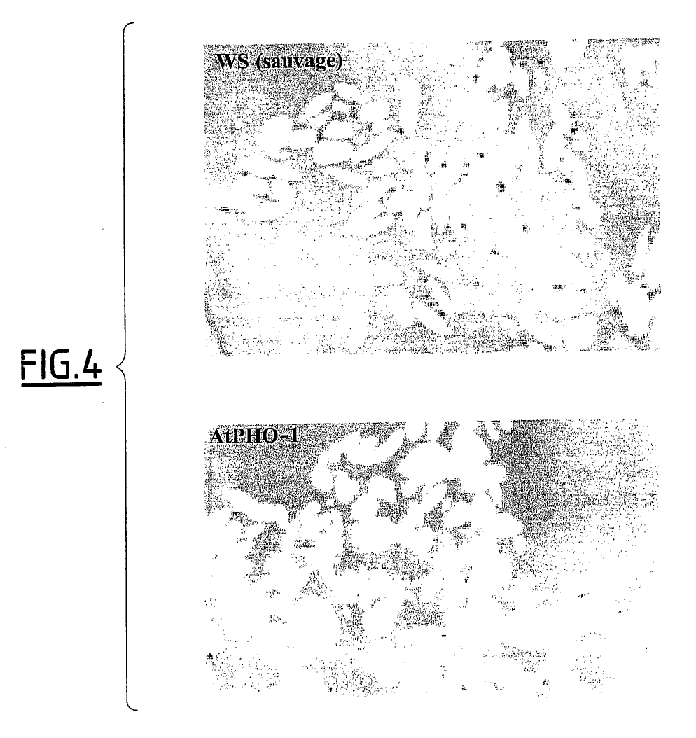

The samples are embedded in 3% agar in water. They are then treated with PATAg: periodic acid-thiosemicarbazide-silver with an incubation time of 20 minutes in periodic acid. They are then embedded in a hydrophilic resin (nanoplast) for 10 days before consolidating the preparation by embedding in LR-White Hard grade resin. The slices are prepared using the ultramicrotome (microme MT-7000) with thickness from 60 to 100 nm. The observations are performed with the TEM (Jeol 100S) at 80 keV (FIG. 4).

The images obtained were analysed for the following parameters:
total area,
equivalent diameter,
ratio of the different lengths.

The values are processed grain by grain.

Regarding the wild-type line, there is considerable size variation: there are numerous quite large grains but also some very small grains. Out of 556 grains analysed, the mean equivalent diameter is 1.27 μm. Grains of elongated shape seem to form the majority.

Regarding the mutant line, the grains are of large size and of more rounded shape (convex) with angular grains present. 256 grains were analysed.

Statistical analysis shows that the starch grains of the mutant line at the AtPHO-1 locus are on average larger than those of the wild-type line.

Thus, two main changes are observed with respect to the starch in the mutant line at the AtPHO-1 locus in A. thaliana:

1) an increase in the average size of the starch grains in the mutant line, 2) a significant increase in the amount of starch accumulated in the leaves.

5. Interaction Between starch Phosphorylase, Starch Synthase, and the Branching Enzymes:

Phosphorylase is one of the first enzymes involved in the biosynthesis of starch; it occurs in the amyloplasts of the endosperm in maize. The enzyme is then present throughout the process of biosynthesis of starch and is the second most abundant enzyme in this process (after the branching enzyme IIb). Studies of zymograms on native gels were able to identify a zone where three different activities are present (soluble starch synthase SSS or SS; branching enzymes SBE, and phosphorylase), suggesting the existence of a complex that includes the enzymes responsible for these activities. Furthermore, enzymatic fractionation coupled with zymograms showed there is interaction between starch phosphorylase and the branching enzymes.

These zymograms employed the following conditions:

The principle of the zymograms is to submit the enzymes to separation by electrophoresis, the electrophoresis gels then being soaked in a solution that triggers the enzymatic reaction where the enzyme has migrated.

For detecting starch phosphorylase, the solution brought into contact with the gel contains glucose 1-phosphate, a substrate of the enzyme. The enzymatic reaction gives rise to the generation and elongation of linear glucan. Blue bands appear where the enzyme has migrated.

For detecting starch synthase, the solution brought into contact with the gel contains amylopectin and ADP-glucose, substrates of the enzyme. The enzymatic reaction gives rise to elongation of the amylopectin chains with the ADP-glucose. Blue bands appear where the enzyme has migrated.

For detecting the branching enzymes (SBE), the solution brought into contact with the gel contains glucose 1-phosphate, a substrate of the enzyme, and an exogenous phosphorylase b (from rabbit). The enzymatic reaction gives rise to generation and elongation of linear glucans with the glucose 1-phosphate, these glucans being branched by the SBE. Brown bands appear where the enzyme has migrated.

Other studies in maize mutants and double transgenic maizes (a/aSBE2b and a/s SSI) showed that the domain of multiple enzymatic activities observed on the native gels was composed of at least SSI, SBE2b and phosphorylase. Without being bound to this theory, it is probable that, in view of the interaction of phosphorylase with the enzymes directly involved in starch biosynthesis, starch phosphorylase is also involved in starch biosynthesis.

In view of the existence of this complex and because phosphorylase appears early relative to AGPase or SSI in the maize endosperm, we can formulate the hypothesis that starch phosphorylase, using glucose 1-phosphate, generates a nascent chain of glucose polymer which would act as a primer for the activities of the enzymes SBE2b and SSI in the amyloplast of maize.

6. Verification of the Subcellular Compartmentation of PHO1:

In addition to PHO1, there is a second phosphorylase in Arabidopsis thaliana: PHO2.

According to bioinformatic predictions, the subcellular localization of PHO2 should be restricted to the cytosol of the cell, whereas PHO1 should be directed to the chloroplast. To confirm the subcellular localization of PHO1, the inventors undertook purification of the chloroplasts, then recorded a zymogram of the phosphorolytic activities, i.e. by monitoring the activity of a cytosolic form of β-amylase (Zeeman et al., 1998) corresponding to the At4g15210 gene (ram-1 gene described in Laby et al., 2001).

a) Purification of the Chloroplasts:

The chloroplasts were purified following a standard protocol:

Plants aged from three to four weeks were left in complete darkness at 4° C. for 48 hours. The leaves (10 g) were collected at 4° C. and homogenized in 200 ml of sorbitol 330 mM, MES 25 mM, pH 6.5, MgCl₂ 5 mM, isoascorbate 2 mM (purification buffer). The homogenate was filtered through three layers of Miracloth and centrifuged for 3 minutes at 2500 g at 4° C. The supernatant corresponds to the cytosol-enriched fraction (CytoEF). The deposit (containing the chloroplasts) was resuspended in 500 μl of the same buffer and loaded on a discontinuous gradient of Percoll: 2 ml of Percoll 65% (bottom of the tube), 2 ml of Percoll 45%, 2 ml of Percoll 20% (top of the tube). The sample was centrifuged for 30 minutes at 4200 g and 4° C. The deposit that formed at the interface between the layers of Percoll at 45% and 65% was collected and diluted in two volumes of the purification buffer and then centrifuged at 1800 g at 4° C. for 2 minutes. The deposit was then washed twice in the same buffer and resuspended in 200 μl of the purification buffer.

b) Zymogram:

The polyacrylamide gel (7.5%) contains rabbit liver glycogen (0.6%). The wells were loaded with 100 μg of proteins and the extract was submitted to migration at 4° C. in native conditions at the rate of 15 mA/gel for 2 hours. The gel was then incubated overnight at room temperature in a buffered medium (sodium citrate 100 mM pH 7.0+glucose 1-phosphate 20 mM). The gel was finally immersed in iodine solution, which reveals the zones where enzymatic activities have altered the structure of the starch (the zones not submitted to the action of modifying enzymes stain orange).

The results confirm that PHO1 is localized in the stroma of the chloroplast whereas PHO2 is an exclusively cytosolic protein.

### BIBLIOGRAPHY

An G. (1986), Plant Physiol. 81: 86-91
Balzergue et al. (2001) Bio Technics, Vol 30, 496-504
Bensen et al. (1995), The Plant Cell, Vol. 7. 75-84
Das et al. (March 1995), The Plant Cell, Vol. 7. 287-294
Depicker et al. (1982) J. Mol. Appl. Genet., 1, 561-573
Finner J. et al. (1992), Plant Cell Reports, 11, 323-328
Fire et al. (1998) Nature 391, 806-811
Franck et al. (1980) Cell. 21, 285-294
Fromm et al. (1986) Nature, vol. 319, 791-793
Gaubier et al. (1993) Mol. Gen., 238, 409-418
Ito et al. (1999) Plant J., Vol 17. 433-44.
Jouanin (1987) Plant. Sci., 53, 53-63
Kay (1987) Science, 236, 1299-1302
Laby et al., (2001) Plant Physiol., 127(4):1798-807
Mc Elroy (1991) Mol. Gen. Genet. 231: 150-160
Ni et al. (1995) Plant J., 7. 661-676
Ratcliff et al. (2001) Plant J. 25, 237-245
Ruiz et al. (1998) Plant Cell 10, 937-946
Sanford J. C., (1988) Trends in Biotechnology, 6, 299-302
Sonnewald et al., (1995) Plant. Mol. Biol. 27. 567-576
Thorneycroft et al., (2001) Journal of Experimental Botany, 52, 361:1593-1601
Waterhouse et al. (1998) Proc. Natl. Acad. Sci. U.S.A. 95, 13959-13964
Watson et al. ADN recombinant (recombinant DNA), Ed. De Boeck Université, p 273-292
Zamore et al., (2000) Cell 101, 25-33 Ecole thématique Biologie végétale—2001
Zeeman et al., (1998) Plant. Cell., 10(10):1699-712

1-9. (canceled)

10. A method for increasing the size of starch grains and/or the starch content of a plant or of a plant part, in which a gene of a starch phosphorylase in cells of the plant is switched off.

11. A method for obtaining plants or plant parts that produce starch grains of increased size or with higher starch content, said method comprising switching off a gene of a starch phosphorylase in cells of the plant.

12. The method according to claim 11, comprising the steps of switching off, by inserting nucleotide(s), a gene coding for said endogenous starch phosphorylase in a plant cell, and regenerating the plant from the transformed cell, said transgenic plant thus obtained having starch grains of increased size, and/or a higher starch content.

13. The method according to claim 10, in which the plant is potato, broad bean, beet, spinach, pea, wheat, maize or rice.

14. A plant cell liable to be obtained by the method according to claim 11.

15. A transgenic plant comprising a plant cell according to claim 14.

16. A seed obtained from the plant according to claim 15, characterized in that it is of increased size, and/or it has an altered starch content.

17. A method for producing a plant with an altered size of starch grains and/or altered starch content, comprising producing said plant with a SEQ ID No2.

18. The method according to claim 17, characterized in that the plant obtained is selected from potato, broad bean, beet, spinach, pea, wheat, maize or rice.
